SEQUENCE LISTING (22 sequences)

SEQ ID NO: 1 - protein, 16 aa, Artificial Sequence
  peptide used for training the SVM model
EQLQDMGLVDLFSPEK

SEQ ID NO: 2 - protein, 13 aa, Artificial Sequence
  peptide used for training the SVM model
VAEGTQVLELPFK

SEQ ID NO: 3 - protein, 10 aa, Artificial Sequence
  peptide used for training the SVM model
LSITGTYDLK

SEQ ID NO: 4 - protein, 10 aa, Artificial Sequence
  peptide used for training the SVM model
SVLGQLGITK

SEQ ID NO: 5 - protein, 15 aa, Artificial Sequence
  peptide used for training the SVM model
DASGVTFTWTPSSGK

SEQ ID NO: 6 - protein, 12 aa, Artificial Sequence
  peptide used for training the SVM model
TFTCTAAYPESK

SEQ ID NO: 7 - protein, 13 aa, Artificial Sequence
  peptide used for training the SVM model
CSTSSLLEACTFR

SEQ ID NO: 8 - protein, 10 aa, Artificial Sequence
  peptide used for training the SVM model
DSGFQMNQLR

SEQ ID NO: 9 - protein, 11 aa, Artificial Sequence
  peptide used for training the SVM model
ATWSGAVLAGR

SEQ ID NO: 10 - protein, 14 aa, Artificial Sequence
  peptide used for training the SVM model
CEGPIPDVTFELLR

SEQ ID NO: 11 - protein, 14 aa, Artificial Sequence
  peptide used for training the SVM model
AESPEVCFNEESPK

SEQ ID NO: 12 - protein, 11 aa, Artificial Sequence
  peptide used for training the SVM model
FTDSENVCQER

SEQ ID NO: 13 - protein, 11 aa, Artificial Sequence
  peptide used for training the SVM model
DLATVYVDVLK

SEQ ID NO: 14 - protein, 13 aa, Artificial Sequence
  peptide used for training the SVM model
DYVSQFEGSALGK

SEQ ID NO: 15 - protein, 9 aa, Artificial Sequence
  peptide used for training the SVM model
ISASAEELR

SEQ ID NO: 16 - protein, 17 aa, Artificial Sequence
  peptide used for training the SVM model
SLAELGGHLDQQVEEFR

SEQ ID NO: 17 - protein, 17 aa, Artificial Sequence
  peptide used for training the SVM model
TTPPVLDSDGSFFLYSR

SEQ ID NO: 18 - protein, 27 aa, Artificial Sequence
  peptide used for training the SVM model
YGPPCPSCPAPEFLGGPSVFLFPPKPK

SEQ ID NO: 19 - protein, 11 aa, Artificial Sequence
  peptide used for training the SVM model
ALGHLDLSGNR

SEQ ID NO: 20 - protein, 10 aa, Artificial Sequence
  peptide used for training the SVM model
DLLLPQPDLR

SEQ ID NO: 21 - protein, 16 aa, Artificial Sequence
  peptide used for training the SVM model
ADGESCSASMMYQEGK

SEQ ID NO: 22 - protein, 8 aa, Artificial Sequence
  peptide used for training the SVM model
IEDGFSLK

(19) United States
(12) Patent Application Publication
(10) Pub. No.: US 2020/0400685 A1
(43) Pub. Date: Dec. 24, 2020

(54) Biomarkers for Typing Allograft Recipients

(71) Applicants: VITO NV, Mol (BE); Katholieke Universiteit Leuven, Leuven (BE); Institut National de La Sante et de la Recherche Medicale (INSERM), Paris (FR); APHP - Assistance Publique - Hôpitaux Paris, Paris (FR); University Hospital Center of Limoges, Limoges (FR); Medizinische Hochschule Hannover, Hannover (DE)

(72) Inventors: Inge Mertens, Mol (BE); Hanny Willems, Mol (BE); Maarten Naessens, Leuven (BE); Pierre Marquet, Paris (FR); Dany Anglicheau, Paris (FR); Marie Essig, Limoges (FR); Wilfried Gwinner, Hannover (DE)

(73) Assignees: VITO NV, Mol (BE); Medizinische Hochschule Hannover, Hannover (DE); Katholieke Universiteit Leuven, Leuven (BE); Institut National de La Sante et de la Recherche Medicale (INSERM), Paris (FR); APHP - Assistance Publique - Hôpitaux Paris, Paris (FR); University Hospital Center of Limoges, Limoges (FR); Medizinische Hochschule Hannover, Hannover (DE)

(21) Appl. No.: 16/958,240

(22) PCT Filed: Dec. 27, 2018
(86) PCT No.: PCT/EP2018/097044

§ 371 Date: Jun. 26, 2020

(30) Foreign Application Priority Data
Dec. 27, 2017 (EP) 17306944.4

(51) Int. Cl. G01N 33/68

(52) U.S. Cl. CPC G01N 33/6893; G01N 2800/245; G01N 2333/7454; G01N 2333/4716; G01N 2333/775; G01N 2333/76; G01N 2333/4728; G01N 2333/81

(57) ABSTRACT

The invention relates to biomarkers for typing or classifying allograft recipients as belonging to a transplant rejection group associated with antibody-mediated rejection (ABMR). The invention also provides for the treatment of typed allograft recipients suffering from antibody-mediated rejection by administration of an appropriate therapeutic agent.

### FIELD OF THE INVENTION

This invention relates to the field of molecular diagnostics, more specifically to the field of biomarkers for typing allograft recipients according to a transplant rejection status, which allows for identification of allograft recipients suffering, or at risk of suffering, from a transplant rejection associated with antibody-mediated rejection (ABMR). This invention is also in the field of therapy, more specifically in the field of treatment of allograft recipients suffering from transplant rejection associated with ABMR, wherein said recipients have been typed or assigned according to a method as described herein.

### STATE OF THE ART

Transplantation of an organ or tissue from a donor to a host patient is part of certain medical procedures and treatment protocols. Despite efforts to avoid allograft rejection through host-donor tissue type matching, in transplantation procedures where a donor organ is introduced into a host, immunosuppressive therapy is generally required to the maintain viability of the donor organ in the host. This means that allograft rejection, or transplant rejection, is a phenomenon that almost always at least to some extent occurs in allograft recipients. One could therefore say that allograft recipients are in principle by default at least to some extent at risk of transplant rejection. Even despite the wide use of immunosuppressive therapy, organ transplant rejection by an alloimmunity response thus occurs.

Alloimmunity-driven rejection of transplants can be divided into transplant rejection mechanisms predominantly driven by either T cell-mediated rejection (TCMR) or antibody-mediated rejection (ABMR).

Assays for monitoring allografts in patients, especially renal allografts, are known in the art. One of the current clinical, non-invasive methods for monitoring kidney allografts is based on the measurement of serum creatinine levels (Rabant M, et al., J Am Soc Nephrol, 26:2840-51 (2015)), the glomerular filtration rate (Wadei H. M. et al., JAm Soc Hypertens., 5(1):39-47 (2011)) and proteinuria (Naesens M. et al. J Am Soc Nephrol, 27:281-92 (2016)). These markers are non-specific and only detect pathologies at a relatively advanced stage. Also, they fail to detect subclinical changes that have not, or do not, surface as a clinical disease. It is therefore not possible to identify an underlying transplant rejection mechanism.

Non-invasive assays in the diagnosis of renal allograft rejection have been proposed. One of such assays relates to the identification of a number of urinary proteins that are indicative for acute transplant rejection (Sigdel et al., Proteomics Clin Appl., 4(1):32-47 (2010)). These markers, however, fail to differentiate between transplant rejection phenotypes, and thus do not allow for a clinical situation wherein allograft recipients suffering, or at risk of suffering, from transplant rejection, are stratified according to their rejection phenotype/mechanism, and thus also do not allow therapy to be tailored in accordance with their specific type of transplant rejection mechanism phenotype/mechanism.

Hitherto, no biomarkers have been developed that are able to classify allograft recipients on the basis of their underlying transplant rejection mechanism. It would be highly advantageous to stratify allograft recipients according to their underlying transplant rejection mechanism, and be able to identify ABMR cases, since this opens up a new clinical situation wherein therapy against transplant rejection associated with ABMR can be tailored to the needs of the allograft recipient. This is especially relevant in the context of long term survival of kidney allografts, where early identification of the underlying rejection mechanism—sometimes even before transplant rejection symptoms surface—and subsequent tailored therapy, are important indicators for long-term survival of the graft.

It is an aim of the invention to provide for biomarkers that allow for the aforementioned classification of allograft recipients, which biomarkers can be sampled in a non-invasive manner, and which provide for good diagnostic performance. In the same context, it is an aim of the invention to allow for early identification of transplant rejection in allograft recipients, stratified according to rejection mechanism, the latter allowing for assignment of personalized therapy tailored to the patient-specific transplant rejection mechanism. Distinguishing ABMR from non-ABMR phenotypes will further mitigate transplant injury and guide optimal immunosuppression dosing.

### THE INVENTION

The present invention solves these problems by providing a method for typing an allograft recipient for the presence or absence of an antibody mediated rejection (ABMR), comprising the steps of—providing a sample comprising proteins from an allograft recipient; —measuring in said sample a protein level for at least two genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDCl73, CYSTM1 and APOA1, preferably as listed in FIG. 1; —comparing said measured protein level to a reference protein level for said at least two genes; and—typing said allograft recipient for the presence or absence of an ABMR on the basis of the comparison of the measured protein level and the reference protein level. In the same manner, the invention provides a method for typing an allograft recipient for the presence or absence of an antibody mediated rejection (ABMR), comprising the steps of
measuring in a sample comprising proteins from an allograft recipient a protein level for at least two genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1; —comparing said measured protein level to a reference protein level for said at least two genes; and—typing said allograft recipient for the presence or absence of an ABMR on the basis of the comparison of the measured protein level and the reference protein level.

Alternatively, the invention solves these problems by providing a method of assigning an allograft recipient to an ABMR group or a non-ABMR group, comprising the steps of: —providing a sample comprising proteins from an allograft recipient suffering, or at risk of suffering, from transplant rejection; —measuring in said sample a protein level for at least two genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1, preferably as listed in FIG. 1; —comparing said measured protein level to a reference protein level for said at least two genes; and—assigning said allograft recipient to said ABMR group or to said non-ABMR group on the basis of the comparison of the measured protein level and the reference protein level. In the same manner, the invention provides a method for assigning an allograft recipient to an ABMR group or a non-ABMR group comprising the steps of: —measuring in a sample comprising proteins from an allograft recipient suffering, or at risk of suffering, from transplant rejection a protein level for at least two genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1; —comparing said measured protein level to a reference protein level for said at least two genes; and—assigning said allograft recipient to said ABMR group or to said non-ABMR group on the basis of the comparison of the measured protein level and the reference protein level.

The inventors discovered a set of 21 genes (listed in FIG. 1), of which protein expression is upregulated in body samples of allograft recipients that exhibit an antibody mediated rejection (ABMR) phenotype—such a phenotype can be determined by histological analysis of an allograft biopsy—as compared to allograft recipients that do not display an ABMR phenotype, which includes (i) recipients of which the allograft is healthy or normal as can be determined by histological analysis of an allograft biopsy, and (ii) recipients of which the allograft displays a rejection phenotype other than an ABMR phenotype, such as T-cell mediated rejection (TCMR), polyomavirus-associated nephropathy (PVAN), interstitial fibrosis and tubular atrophy (IFTA), glomerulonephritis (GNF) or combinations thereof, as can all be determined by histological analysis of an allograft biopsy. The discovered biomarkers can individually be used in typing ABMR. In addition, biomarkers from said set have been shown to provide good diagnostic performance and were successfully validated in an independent cohort of patients suffering from kidney failure which previously received a kidney allograft (Tables 2-3 and 4-7; and FIGS. 2 and 4-6).

The term “typing”, as used herein, refers to differentiating between, or stratification of, allograft recipients according to a transplant rejection (sub)class. The typing is based on a comparison of (i) the measured protein level for at least one or at least two genes listed in FIG. 1, with (ii) a reference protein level for said at least one or at least two genes. In particular, the typing is based on a comparison of (i) the measured protein level for at least two genes listed in FIG. 1, with (ii) a reference protein level for said at least two genes.

The term “allograft”, as used herein, refers to an organ or tissue transplant that is transplanted from one individual or subject to another of the same species with a different genotype. The term “allograft” can also be referred to as “allogenic graft”. Unless the context clearly dictates otherwise, the terms “allograft” and “transplant”, as used herein, refer to the object of transplantation (noun). Allografts are provided by donors and can be from a living or cadaveric source. Preferably, the allograft is an organ selected from the group formed by heart, kidney, liver, lung, pancreas, intestine or thymus. Alternatively, the allograft can be a tissue, such as bone, tendon (both referred to as musculoskeletal grafts), corneae, skin, heart valve, nerve or veins. The terms “allograft” and “transplant” are used interchangeably herein, unless the context dictates otherwise.

Preferably, in a method of typing or assigning of the invention (a method of the invention), the allograft is a kidney allograft, which may also be referred to as a renal allograft herein.

Preferably, a method of typing or assigning of the invention is an in vitro method.

The term “allograft recipient”, as used herein, refers to a subject, preferably a mammal, more preferably a primate, most preferably a human, that has received an allograft. Unless the context clearly dictates otherwise, the term “allograft recipient”, as used herein, refers to a subject or individual that has already received the allograft through transplantation. Preferably, the allograft recipient suffers, or previously suffered, from organ failure which necessitated transplantation of an organ or tissue allograft. In other words, preferably, the allograft recipient is a subject or individual which received an organ or tissue allograft through transplantation for treating organ or tissue failure.

For the purpose of this disclosure, organ failure is considered to be organ dysfunction to such a degree that normal homeostasis cannot be maintained without clinical intervention in the form of a organ or tissue transplantation.

Preferably, the allograft recipient suffers, or previously—before transplantation—suffered, from kidney failure which was treated by transplantation of a kidney allograft. In other words, preferably, the allograft recipient is a subject or individual which received a kidney allograft through transplantation for treating kidney failure. The term “kidney failure”, as used herein, may also be referred to as end-stage renal disease.

The present invention allows for differentiating allograft recipients according to a transplant rejection (sub)class. As to date, no such “deep” characterization of a transplanted allograft has been possible with methods other than histological analysis of a biopsy (invasive) of the transplanted allograft. In addition, the results in the Examples indicate that a patient population currently not identified through biopsy analysis is picked up when using a method of the invention, allowing for improvement in current therapy.

The term “transplant rejection”, as used herein, refers to a disease condition caused by the recipient's or host's immune system in response to a transplanted allograft, which can damage or destroy the allograft. One of skill in the art thus realizes that the condition of transplant rejection is controlled by the allograft recipient. The term explicitly covers all stages of transplant rejection, including subclinical transplant rejection and clinical transplant rejection. The term “subclinical rejection” or “subclinical transplant rejection”, as used herein, refers to a disease condition which is not severe enough to present definite or readily observable symptoms, but where histologic evidence of rejection on an allograft biopsy is preferably, but not necessarily, found, optionally without an elevation in the serum creatinine concentration. Subclinical transplant rejection can be one of the factors that contribute to graft loss in the long run.

The term “transplant rejection”, as used herein, encompasses both acute and chronic (transplant) rejection.

The terms “acute (transplant) rejection” or “AR”, as used herein, refer to the rejection of a transplant by the immune system of a transplant recipient when the transplanted tissue is immunologically foreign. Acute rejection is characterized by infiltration of the transplant by immune cells of the recipient, or effectors thereof, which may damage or destroy the transplant. The onset of acute rejection is rapid and generally occurs in humans within a few weeks after transplant surgery. Generally, acute rejection can be inhibited or suppressed with immunosuppressive drugs such as rapamycin, everolimus, cyclosporin, tacrolimus, mycophenolic acid, anti-CD25 monoclonal antibody and the like. The term “acute (transplant) rejection” covers inter alia both acute (or active) antibody mediated rejection (ABMR) and acute T-cell mediated rejection (TCMR).

The term “chronic (transplant) rejection”, as used herein, refers to a disease condition that generally occurs in humans within several months to years after engraftment of the transplant, even in the presence of successful immunosuppression of acute (transplant) rejection. Fibrosis is a common factor in chronic rejection of all types of organ transplants. Chronic rejection can typically be described by a range of specific disorders that are characteristic of a particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerosis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs. The term “chronic transplant rejection” inter alia covers both chronic ABMR and chronic TCMR.

Preferably, in a method for typing, assigning or measuring according to the invention, the allograft recipient suffers from a transplant rejection—which suffering may explicitly encompasses subclinical rejection—or is at risk of suffering from transplant rejection. In principle, the allograft recipient is per definition at risk of suffering from transplant rejection, because the graft is allogeneic. It is evident that, in the context of the invention, the term “suffering” does not mean that symptoms of transplant rejection are already apparent to the allograft recipient. For that reason, the term also encompasses transplant rejection that is subclinical. The terminology “suffering or suffers from transplant rejection” may also be rephrased as “undergoing a transplant rejection response”.

Preferably, in a method of the invention, the transplant rejection is an acute (transplant) rejection, and the ABMR or TCMR associated with said acute transplant rejection are consequently preferably an acute ABMR or an acute TCMR.

The phrase “transplant rejection associated with an ABMR”, as used herein, includes reference to a transplant rejection that is at least to some extent, but preferably predominantly, driven by ABMR, and said phrase may also be rephrased by simply using the term “ABMR” or the phrase “transplant rejection that is ABMR”. A corresponding phrasing applies where the referenced rejection phenotype is a non-ABMR such as TCMR.

ABMR is an often severe form of allograft rejection. The pathophysiology of ABMR suggests a prime role for antibodies, B-cells and plasma cells, but other effector molecules, especially the complement system, point to potential targets that could modify the ABMR process. ABMR continues to be observed in 30-40% cases of kidney transplant cases, comprising the primary cause of early graft loss. The skilled person is aware of methods and means to determine whether an allograft biopsy is subjected to ABMR, such as for instance by performing a histological analysis using the Banff classification categories such as the updated 2015 Banff classification categories as reported on below.

In allografts, TCMR is characterized by infiltration of the interstitium by T cells and macrophages, intense IFNγ and TGFB® effects, and epithelial deterioration.

ABMR and TCMR of an allograft are generally identified according the generally known Banff classification categories, such as the updated 2015 Banff classification categories, of which the sections for acute ABMR and acute TCMR are reproduced herein below. The skilled person is aware that similar guidance for the classification of chronic ABMR, chronic TCMR and interstitial fibrosis and tubular atrophy (IFTA) is provided in these guidelines. The skilled person is further aware that polyomavirus-associated nephropathy (PVAN) and glomerulonephritis (GNF) are classified in a separate rejection category, which can be termed “Other changes not considered to be caused by acute or chronic rejection” as is inter alia described in Loupy et al., 2017 (Loupy et al., Am J Transplant., 17(1):28-41 (2017)).

### Updated 2015 Banff Classification Categories

### Category 2: Antibody-Mediated Changes

Acute/active ABMR: All three features must be present for diagnosis. Biopsies showing histological features plus evidence of current/recent antibody interaction with vascular endothelium or DSA, but not both, may be designated as suspicious for acute/active ABMR. Lesions may be clinically acute or smoldering or may be subclinical; it should be noted if the lesion is C4d-positive or C4d-negative, based on the following criteria:

1. Histologic evidence of acute tissue injury, including one or more of the following:
Microvascular inflammation (g >0 in the absence of recurrent or de novo glomerulonephritis, and/or ptc >0)
Intimal or transmural arteritis (v >0)
Acute thrombotic microangiopathy in the absence of any other cause
Acute tubular injury in the absence of any other apparent cause
   2. Evidence of current/recent antibody interaction with vascular endothelium, including at least one of the following:
Linear C4d staining in peritubular capillaries (C4d2 or C4d3 by IF on frozen sections or C4d >0 by IHC on paraffin sections)
At least moderate microvascular inflammation ([g+ptc]>2), although in the presence of acute TCMR, borderline infiltrate, or infection; ptc >2 alone is not sufficient, and g must be >1
Increased expression of gene transcripts in the biopsy tissue indicative of endothelial injury, if thoroughly validated
   3. Serologic evidence of DSAs (HLA or other antigens)
Biopsies suspicious for ABMR on the basis of meeting criteria 1 and 2 should prompt expedited DSA testing

### Updated 2015 Banff Classification Categories

### Category 4: Acute TCMR (Grade)

IA. Significant interstitial inflammation (>25% of nonsclerotic cortical parenchyma, i2 or i3) and foci of moderate tubulitis (t2)
IB. Significant interstitial inflammation (>25% of nonsclerotic cortical parenchyma, i2 or i3) and foci of severe tubulitis (t3)
IIA. Mild to moderate intimal arteritis (v1) with or without interstitial inflammation and tubulitis
IIB. Severe intimal arteritis comprising >25% of the luminal area (v2) with or without interstitial inflammation and tubulitis
III. Transmural arteritis and/or arterial fibrinoid change and necrosis of medial smooth muscle cells with accompanying lymphocytic inflammation (v3)
In short, T cell mediated rejection (TCMR) can be diagnosed by scoring interstitial inflammation (i), tubulitis (t), and vasculitis (v), while a hallmark of antibody-mediated rejection (ABMR) is C4d deposition in peritubular capillaries.

When the term “ABMR” is employed herein, ABMR of the allograft of an allograft recipient is meant. In the same manner, when the terms “non-ABMR” or “TCMR” are employed herein, non-ABMR or TCMR of the allograft of an allograft recipient is meant, respectively.

Preferably, the ABMR is an ABMR as classified according to the Banff classification methodology. Preferably, the TCMR or IFTA is a TCMR or IFTA, respectively, as classified according to the Banff classification methodology.

Since the terms “ABMR”, “non-ABMR” and “TCMR”, are also used in relation to the term “phenotype” in the art, these terms can also be referred to as “ABMR phenotype”, “non-ABMR phenotype” and “TCMR phenotype”.

Most preferably, in a method of the invention, the ABMR is an antibody mediated renal rejection (ABMRR), and/or the TCMR is an T-cell mediated renal rejection (TCMRR).

The term “sample”, as used herein, refers to a sample obtained from an allograft recipient, said sample containing proteins. The sample is preferably a body fluid sample. Such samples include, but are not limited to, sputum, blood, serum, plasma, urine, peritoneal fluid and pleural fluid. Most preferably, the sample is a urine sample. Obtaining such samples is well within common general knowledge of the skilled person. This term also includes reference to processed samples, for instance samples that are prepared for undergoing a protein level measurement step.

Preferably, a method of the invention is for (i) typing a sample of said allograft recipient for the presence or absence of an ABMR, or (ii) assigning a sample of said allograft recipient to an ABMR group or a non-ABMR group.

In another step of a method of the invention, in said sample a protein level, also referred to as protein expression level, is measured or determined for at least two genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1. It is clear to the skilled person that, when reference is made to protein levels that are measured for genes, it is intended to refer to measurement of the protein expression product that is ultimately produced by transcription of the gene and translation of the gene transcription product.

The terms “protein” and “peptide”, as used herein, refer to a polymer of amino acid residues (an amino acid sequence) and does not refer to a specific length of the molecule. This term also refers to or includes any modifications of the polypeptide (e.g., post-translational), such as glycosylations, acetylations, phosphorylations and the like. Included within the definition are, for example, naturally occurring variants of the proteins identified in FIG. 1 by their respective UniProtKB Acc. Nos. In the context of protein levels, the terms protein and peptide are interchangeable.

Preferably, a protein level is measured for at least 2, 3, 4, 5, 6, 7, 8, 9, 10, 11, 12, 13, 14, 15, 16, 17, 18, 19 or at least 21 genes selected from the genes listed in FIG. 1; or a protein level is measured for at least 2, 3, 4, 5, 6, 7, 8, 9, 10, 11, 12, 13 or at least 14 genes selected from TF, SERPINA1, AZGP1, ORM1, ORM2, SERPINC1, IGHA1, IGHG4, TFAP2C, CARD6, SERPINA7, CCDC73, CYSTM1 and APOA1; or a protein level is measured for at least 2, 3, 4, 5, 6, 7, 8, 9, or at least 10 genes selected from TF, SERPINA1, SERPINC1, LRG1, IGHA1, IGHG4, APOA4, AFM, A1BG and APOA1. Preferably, in a method of the invention, a protein level is measured for at least 6 genes selected from TF, SERPINA1, SERPINC1, LRG1, IGHA1, IGHG4, APOA4, AFM, A1BG and APOA1. Preferably, in a method of the invention, a protein level is measured for at least genes TF and SERPINA1, more preferably for at least genes TF, SERPINA1 and APOA4, even more preferably for at least genes TF, SERPINA1, APOA4 and AZGP1 optionally in each of said embodiments further supplemented with ORM1, ORM2, C3, A1BG and/or SERPINC1. Alternatively, a protein level is measured for at least the, from top (TF) to bottom (CYSTM1), first 2, 3, 4, 5, 6, 7, 8, 9, 10, 11, 12, 13, 14, 15, 16, 17, 18, 19 20 or 21 genes selected from the genes listed in FIG. 1.

Alternatively, a protein level is measured for at least genes TF, SERPINA1, AFM, A1BG, SERPINC1 and IGHA1.

It is preferred that in a method of the invention as described herein, a protein level is measured for at least two genes selected from the group formed by A1BG, AFM, APOA1, APOA4, IGHA1, IGHG4, LRG1, SERPINA1, SERPINC1 and TF. For that matter, it is preferred that in a method as described herein, a protein level is measured for at least genes A1BG and AFM; A1BG and APOA1; A1BG and APOA4; A1BG and IGHA1; A1BG and IGHG4; A1BG and LRG1; A1BG and SERPINA1; A1BG and SERPINC1; A1BG and TF; AFM and APOA1; AFM and APOA4; AFM and IGHA1; AFM and IGHG4; AFM and LRG1; AFM and SERPINA1; AFM and SERPINC1; AFM and TF; APOA1 and APOA4; APOA1 and IGHA1; APOA1 and IGHG4; APOA1 and LRG1; APOA1 and SERPINA1; APOA1 and SERPINC1; APOA1 and TF; APOA4 and IGHA1; APOA4 and IGHG4; APOA4 and LRG1; APOA4 and SERPINA1; APOA4 and SERPINC1; APOA4 and TF; IGHA1 and IGHG4; IGHA1 and LRG1; IGHA1 and SERPINA1; IGHA1 and SERPINC1; IGHA1 and TF; IGHG4 and LRG1; IGHG4 and SERPINA1; IGHG4 and SERPINC1; IGHG4 and TF; LRG1 and SERPINA1; LRG1 and SERPINC1; LRG1 and TF; SERPINA1 and SERPINC1; SERPINA1 and TF; or SERPINC1 and TF. Highly preferred are (i) at least A1BG and APOA4, (ii) A1BG and SERPINA1, (iii) A1BG and TF, (iv) APOA4 and SERPINA1, (v) APOA4 and TF or (vi) SERPINA1 and TF.

Alternatively, a protein level is measured for at least genes AZGP1 and TF; AZGP1 and SERPINA1; AZGP1 and APOA4; AZGP1 and AFM; AZGP1 and ORM1; AZGP1 and ORM2; AZGP1 and C3; AZGP1 and A1BG; AZGP1 and SERPINC1; AZGP1 and LRG1; AZGP1 and IGHA1; AZGP1 and IGHG4; AZGP1 and TFAP2C; AZGP1 and HPX; AZGP1 and A2M; AZGP1 and CARD6; AZGP1 and SERPINA7; AZGP1 and CCDC73; AZGP1 and CYSTM1; AZGP1 and APOA1; ORM1 and TF; ORM1 and SERPINA1; ORM1 and APOA4; ORM1 and AFM; ORM1 and ORM2; ORM1 and C3; ORM1 and A1BG; ORM1 and SERPINC1; ORM1 and LRG1; ORM1 and IGHA1; ORM1 and IGHG4; ORM1 and TFAP2C; ORM1 and HPX; ORM1 and A2M; ORM1 and CARD6; ORM1 and SERPINA7; ORM1 and CCDC73; ORM1 and CYSTM1; ORM1 and APOA1; ORM2 and TF; ORM2 and SERPINA1; ORM2 and APOA4; ORM2 and AFM; ORM2 and C3; ORM2 and A1BG; ORM2 and SERPINC1; ORM2 and LRG1; ORM2 and IGHA1; ORM2 and IGHG4; ORM2 and TFAP2C; ORM2 and HPX; ORM2 and A2M; ORM2 and CARD6; ORM2 and SERPINA7; ORM2 and CCDC73; ORM2 and CYSTM1; ORM2 and APOA1; C3 and TF; C3 and SERPINA1; C3 and APOA4; C3 and AFM; C3 and A1BG; C3 and SERPINC1; C3 and LRG1; C3 and IGHA1; C3 and IGHG4; C3 and TFAP2C; C3 and HPX; C3 and A2M; C3 and CARD6; C3 and SERPINA7; C3 and CCDC73; C3 and CYSTM1; C3 and APOA1; TFAP2C and TF; TFAP2C and SERPINA1; TFAP2C and APOA4; TFAP2C and AFM; TFAP2C and A1BG; TFAP2C and SERPINC1; TFAP2C and LRG1; TFAP2C and IGHA1; TFAP2C and IGHG4; TFAP2C and HPX; TFAP2C and A2M; TFAP2C and CARD6; TFAP2C and SERPINA7; TFAP2C and CCDC73; TFAP2C and CYSTM1; TFAP2C and APOA1; HPX and TF; HPX and SERPINA1; HPX and APOA4; HPX and AFM; HPX and A1BG; HPX and SERPINC1; HPX and LRG1; HPX and IGHA1; HPX and IGHG4; HPX and A2M; HPX and CARD6; HPX and SERPINA7; HPX and CCDC73; HPX and CYSTM1; HPX and APOA1; A2M and TF; A2M and SERPINA1; A2M and APOA4; A2M and AFM; A2M and A1BG; A2M and SERPINC1; A2M and LRG1; A2M and IGHA1; A2M and IGHG4; A2M and CARD6; A2M and SERPINA7; A2M and CCDC73; A2M and CYSTM1; A2M and APOA1; CARD6 and TF; CARD6 and SERPINA1; CARD6 and APOA4; CARD6 and AFM; CARD6 and A1BG; CARD6 and SERPINC1; CARD6 and LRG1; CARD6 and IGHA1; CARD6 and IGHG4; CARD6 and SERPINA7; CARD6 and CCDC73; CARD6 and CYSTM1; CARD6 and APOA1; SERPINA7 and TF; SERPINA7 and SERPINA1; SERPINA7 and APOA4; SERPINA7 and AFM; SERPINA7 and A1BG; SERPINA7 and SERPINC1; SERPINA7 and LRG1; SERPINA7 and IGHA1; SERPINA7 and IGHG4; SERPINA7 and CCDC73; SERPINA7 and CYSTM1; SERPINA7 and APOA1; CCDC73 and TF; CCDC73 and SERPINA1; CCDC73 and APOA4; CCDC73 and AFM; CCDC73 and A1BG; CCDC73 and SERPINC1; CCDC73 and LRG1; CCDC73 and IGHA1; CCDC73 and IGHG4; CCDC73 and CYSTM1; CCDC73 and APOA1; CYSTM1 and TF; CYSTM1 and SERPINA1; CYSTM1 and APOA4; CYSTM1 and AFM; CYSTM1 and A1BG; CYSTM1 and SERPINC1; CYSTM1 and LRG1; CYSTM1 and IGHA1; CYSTM1 and IGHG4; CYSTM1 and APOA1.

It is shown that the protein levels of at least two genes from the genes listed in FIG. 1 already allow for typing of ABMR (FIG. 6).

More preferably, the protein level is measured for at least one or at least two genes selected from the group formed by A1BG, APOA4, SERPINA1 and TF. Even more preferably the protein level is measured for at least 6 genes selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1; even more preferably, said at least 6 genes are selected from the group formed by A1BG, AFM, APOA1, APOA4, IGHA1, IGHG4, LRG1, SERPINA1, SERPINC1 and TF, such as (i) at least A1BG, APOA1, APOA4, IGHA1, SERPINA1 and TF, (ii) at least A1BG, APOA4, IGHA1, LRG1, SERPINA1 and TF or (iii) at least A1BG, APOA1, APOA4, LRG1, SERPINA1 and TF.

Preferably, in a method of the invention, a protein level is measured for at least genes TF and SERPINA1, more preferably for at least genes TF, SERPINA1 and APOA4, even more preferably for at least genes TF, SERPINA1, APOA4 and A1BG optionally in each of said embodiments further supplemented with AFM, APOA1, IGHA1, IGHG4, LRG1 and/or SERPINC1.

The invention also provides for a method for typing an allograft recipient for the presence or absence of an antibody mediated rejection (ABMR), comprising the steps of—measuring in a sample comprising proteins from an allograft recipient a protein level for at least one gene selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1; —comparing said measured protein level to a reference protein level for said at least one gene; and—typing said allograft recipient for the presence or absence of an ABMR on the basis of the comparison of the measured protein level and the reference protein level. FIG. 1 shows that protein levels of all individual genes are correlated to AMBR. The invention also provides for a method for assigning an allograft recipient to an ABMR group or a non-ABMR group comprising the steps of: —measuring in a sample comprising proteins from an allograft recipient suffering, or at risk of suffering, from transplant rejection a protein level for at least one gene selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1; —comparing said measured protein level to a reference protein level for said at least one gene; and—assigning said allograft recipient to said ABMR group or to said non-ABMR group on the basis of the comparison of the measured protein level and the reference protein level. The embodiments described herein relating to methods for typing or assigning on the basis of at least two genes, equally apply to such methods employing at least one gene, when appropriate.

The invention also provides a use of at least two (different) proteins, preferably protein levels of said at least two proteins, encoded by genes TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 or APOA1 in a urine sample as a (bio)marker for the presence or absence of an ABMR in an renal allograft recipient. Preferably, the use involves at least two proteins, preferably protein levels of said at least two proteins, encoded by genes A1BG, AFM, APOA1, APOA4, IGHA1, IGHG4, LRG1, SERPINA1, SERPINC1 and TF; more preferably, the use involves at least six proteins, preferably protein levels of said at least six proteins, encoded by genes A1BG, AFM, APOA1, APOA4, IGHA1, IGHG4, LRG1, SERPINA1, SERPINC1 and TF. Embodiments relating to methods as described herein also apply to the use described herein, when appropriate, for instance regarding combinations of proteins/genes.

The skilled person has ample well known methods and means at his disposal for measuring protein or peptide levels for genes in a sample, including measurement of relative or absolute protein or peptide concentrations, and/or longitudinal (multiple sampling of the same patient over time) or cross-sectional (a single time point measurement per patient) measurements.

Exemplary methods for protein or peptide analysis include, but are expressly not limited to, High-performance liquid chromatography (HPLC); mass spectrometry (MS), preferably set up in MS/MS mode; LC-MS based peptide profiling, preferably HPLC-MS, the latter preferably set up in MS/MS mode (shotgun mode/data dependent acquisition (DDA), data independent acquisition (DIA), targeted mode (selected reaction monitoring (SRM), parallel reaction monitoring (PRM), multiple reaction monitoring (MRM)); enzyme-linked immunosorbent assay (ELISA); protein microarray, protein QPCR and the like. In the broadest sense, protein expression evaluation may be qualitative or quantitative. In the present invention, the methods provide for a quantitative detection of whether the protein or peptide is present in the sample being assayed, i.e., an evaluation or assessment of the actual amount or relative abundance of the protein or peptide in the sample being assayed. In such embodiments, the quantitative detection may be absolute or, if the method is a method of detecting two or more different proteins or peptides in a sample, relative. As such, the term “level” or “quantifying” when used in the context of quantifying, or measuring a protein level of, a protein or peptide in a sample can refer to absolute or to relative quantification. Absolute quantification may be accomplished by inclusion of known concentration(s) of one or more control analytes and referencing the detected level of the target protein or peptide with the known control analytes (e.g., through generation of a standard curve). Alternatively, relative quantification can be accomplished by comparison of detected levels or amounts between two or more different target proteins or peptides to provide a relative quantification of each of the two or more different proteins or peptides, e.g., relative to each other. In addition, a relative quantitation may be ascertained using a control, or reference, value (or profile) from one or more control sample. The term “protein level” also encompasses peptide levels, especially where such peptide levels are in fact used as a measure for a protein level. In the same manner, the term “protein” also encompasses protein parts.

In a method of the invention, any convenient protein or peptide quantitation protocol can be employed, where a protein expression level for at least two genes listed in FIG. 1 is measured in the provided sample so as to generate a protein or peptide signature or profile for the sample. Such methods include standard immunoassays including antibody- or aptamer-based protein quantification assays (e.g., ELISA assays, such as a multiplex ELISA assay, Western blots, FACS-based protein analysis, and the like), protein activity assays, including multiplex protein activity assays, protein QPCR, protein expression arrays, etc.

A method of the invention may further comprise the steps of: —digesting proteins in said sample with trypsin (or any alternative protease or combinations thereof) so as to provide a mixture of peptides; —subjecting said mixture of peptides to a step of liquid chromatography (or analogous separation techniques like capillary electrophoresis) so as to provide an eluate comprising peptides; and—performing a step of mass spectrometry on said eluate to measure a peptide level for at least two peptides, said peptide level for said at least two peptides representing the protein level for said at least two genes.

The skilled person understands that the peptide level for said at least two genes is a measure for the protein level for said at least two genes as referred to in a method of the invention. In addition, performance of a step of mass spectrometry as referred to above may be further specified by measuring or providing a peptide profile/signature, and measuring, or determining, a peptide level for at least two peptides, said peptide level for said at least two peptides representing, or being a measure for, the protein level for said at least two genes. It is clear to the skilled person that, with this phrasing, it is intended that each one of said at least two peptides originally formed part of a protein encoded by a different gene of said at least two genes. The skilled person understands that a peptide level for a further peptide can be measured or determined, and that such a peptide can represent the same protein as one of the first two peptides, or instead be an identifier for a further protein listed in FIG. 1.

In the same context, the present inventors identified a set of twelve unique peptides (Table 1, SEQ ID Nos: 1-12) that are specific identifiers for six proteins listed in FIG. 1 (green/grey marked proteins). In the same manner, ten further peptides (SEQ ID Nos: 13-22) were identified (Table 4). When MS is employed as protein or peptide measurement tool, these peptide sequences provides for the benefit that peaks in the generated MS profile corresponding to these peptides can be easily identified and attributed to a protein biomarker as described herein. MS peaks of such a peptide is a measure for its peptide level, and given the fact that these peptides are unique for a protein listed in FIG. 1, they also provide a measure for the protein level. It should however be understood that protein expression levels can be measured by numerous other methods.

Thus, preferably, in a method of the invention, the at least two peptides are selected from peptides having a sequence of SEQ ID NOs:1-12 and/or 13-22. More preferably, in a method of the invention, a peptide level of at least 3, 4, 5, 6, 7, 8, 9, 10, 11 or 12 of the peptides listed in Table 1 is measured or determined or at least 2, 3, 4, 5, 6, 7, 8, 9, 10, 11, 12, 13, 14, 15, 16, 17, 18, 19, 20 of the peptides listed in Table 4 is measured or determined. Even more preferably, a peptide level is determined for at least two peptides selected from (i) SEQ ID NO: 3 or 4; (ii) SEQ ID NO: 7 or 8; and (iii) SEQ ID NO: 11 or 12.

Alternatively, in a method of the invention, measurement of a protein level is performed by an enzyme-linked immunosorbent assay (ELISA).

A method of typing or assigning of the invention further comprises a step of comparing the measured, or determined, protein level to a reference protein level.

After measuring and determining the protein level of the target proteins, and for instance providing such protein level data in the form of a profile or signature, the protein level is analyzed or evaluated to determine whether the allograft recipient is suffering from, or undergoing, an ABMR or non-ABMR response. Such an analysis involves comparison of the measured, or determined, protein level for a set of genes to a reference protein level for the same set of genes.

The term “reference protein level” denotes a standardized protein level (or standardized protein level profile or signature, or total normalized protein level) that can be used to interpret the protein level measured, or determined, in a sample of an allograft recipient.

A reference protein level that is appropriate for typing or assigning purposes of the present invention can be set by a skilled person in multiple, alternatives ways, such setting of reference protein levels belonging to common general knowledge of the skilled person.

For instance, in a method of the invention, a reference protein level can be a reference protein level of said at least two genes in a reference sample, preferably obtained on the basis of a reference sample. The reference sample can be a sample from any individual, such as a healthy or diseased individual, but is preferably a sample from allograft recipient. The reference sample from an allograft recipient can be a sample from a healthy allograft recipient not showing any signs of transplant rejection, but can also be a reference sample from an allograft recipient suffering from, or undergoing, a transplant rejection (response) such as ABMR or TCMR. The reference sample can also be from an allograft recipient suffering from, or undergoing, a transplant rejection (response) that is not an ABMR, but instead is a non-ABMR such as TCMR. In a method of the invention, when comparison is made to a reference protein level for said at least two genes in a reference sample from an allograft recipient not having an ABMR, the allograft recipient that is to be typed, or that is to assigned to a group, is typed as having an ABMR or undergoing an ABMR response, or assigned to the ABMR group, respectively, if protein levels are increased as compared to the reference protein level. It was established that the discovered biomarkers in FIG. 1 are upregulated at the protein level when ABMR is present as compared to when its absent. Knowing the protein expression direction, the skilled person can perform a method for typing or assigning as described herein by routinely applying appropriate reference protein levels that either represent similarity or dissimilarity to an AMBR phenotype. Preferably, in any one of the methods described herein, when said recipient is typed as having an ABMR, the protein expression level for said at least two genes is increased or upregulated as compared to the protein expression level of an allograft recipient not having an ABMR.

The skilled person will understand that also multiple reference samples can be used for setting appropriate reference values, such as a sample from one or more allograft recipient(s) having ABMR, and a sample from one or more allograft recipient(s) not having an ABMR, but with the same or different non-ABMR.

The reference sample can also be a pooled protein sample from multiple individuals, preferably allograft recipients as mentioned hereinabove such as allograft recipients not having, or not undergoing, an ABMR (response). Said sample can be pooled from more than 10 individuals, more than 20 individuals, more than 30 individuals, more than 40 individuals or more than 50 individuals.

A highly beneficial reference protein level is an absolute protein level for discriminating ABMR from non-ABMR. It is within the common knowledge of the skilled person to set such an absolute threshold protein level.

Typing of an allograft recipient, or assigning an allograft recipient to a rejection phenotype group, can be performed in various ways. In one method, a coefficient is determined that is a measure of a similarity or dissimilarity of the protein level in a target sample with a previously established reference protein level—for the genes listed in FIG. 1—that can be specific to a certain cell type, tissue, disease state or any other interesting biological or clinical interesting samples group. Such a reference protein expression level can be referred to as a profile template. Typing, or assigning, of a sample can be based on its (dis)similarity to a single profile template or preferably based on multiple profile templates. By determining a correlation with a profile template an overall similarity score for the set of genes can be set. A similarity score is a measure of the average correlation of protein levels of a set of genes in a sample from an allograft recipient and a profile template. Said similarity score can, but does not need to be, a numerical value between +1, indicative of a high correlation between the protein level of the set of genes in a sample of said allograft recipient and said profile template, and −1, which is indicative of an inverse correlation. A threshold value can then be set to differentiate between samples on the basis of rejection phenotype. Said threshold is an arbitrary value that allows for discrimination between samples from allograft recipients with ABMR, and samples of allograft recipients without ABMR. If a similarity threshold value is employed, it is preferably set at a value at which an acceptable number of allograft recipients with ABMR would score as false negatives, and an acceptable number of patients without ABMR would score as false positives. A similarity score is preferably displayed or outputted to a user interface device, a computer readable storage medium, or a local or remote computer system.

A classic method for calculating a similarity score when having different predictors is linear logistic regression, but there are further statistical and data mining classification methods available to the skilled person that can be used to calculate similarity scores. For instance, a non-limiting example is a support vector machine, which is a statistical learning method for building classification models (Cristianini et al., An Introduction to Support Vector Machines and Other Kernel-based Learning Methods., 2000, Cambridge University Press; Vapnik, The Nature of Statistical Learning Theory., 1995 New York Springer; Zhang et al., BMC Bioinformatics, 7:197 (2006)).

More preferably, in a method of the invention, the reference protein level is a standardized, absolute protein level value, set for antibody- or aptamer-based protein quantification assays such as enzyme-linked immunosorbent assay (ELISA) protein measurements. Such a protein level value functions as a threshold value, allowing for discrimination between protein levels in a sample of an allograft recipient having, suffering from, or undergoing, ABMR, and protein levels in a sample of an allograft recipient not having, not suffering from, or not undergoing ABMR (non-ABMR), but instead for instance TCMR.

In a method of typing of the invention, when the allograft recipient is typed as not having, or not undergoing, an ABMR, the recipient is preferably typed as having a healthy or normal allograft, or an allograft associated with a transplant rejection phenotype that is TCMR.

The term “non-ABMR”, as used herein, includes reference to (i) allografts that are healthy or normal, and thus do not show rejection signs, and (ii) allografts that are associated with a transplant rejection phenotype that is not ABMR, but is instead for instance TCMR, polyomavirus-associated nephropathy (PVAN), interstitial fibrosis and tubular atrophy (IFTA), glomerulonephritis (GNF), or combinations thereof.

Further, a method of the invention may further comprise the step of: —assigning therapy to said allograft recipient; wherein, if said recipient is typed as having, suffering from, or undergoing, ABMR, a standard-of-care therapeutic agent against ABMR as described below is assigned as therapy. A corresponding step can be performed in relation to a method of assigning according to the invention. Alternatively, if said recipient is typed as not having, suffering from, or undergoing, ABMR, a further typing or classification step to identify the underlying non-ABMR phenotype can be performed. A corresponding step can be performed in relation to a method of assigning according to the invention.

In addition, a method of the invention may further comprise a step of: —measuring in a serum sample of said allograft recipient a serum creatinine level; —determining the glomerular filtration rate, preferably by intravenously injecting (i) inulin, (ii) inulin-analogs such as sinistrin or (iii) radioactive substances for use in determining glomerular filtration rate, such as 51Cr-EDTA or 99mTc-DTPA, into the bloodstream of an allograft recipient and measuring its clearance; and/or assaying a urine sample of the allograft recipient for proteinuria by performing a dipstick test or determining a human serum albumin (HSA) level through the use of liquid crystals and comparing such a HSA level to a reference value. Alternatively, the invention also provides a method for measuring protein levels in a sample of a human subject, comprising the steps of:
providing a sample comprising proteins from a human subject, preferably a sample from an human allograft recipient;
measuring in said sample a protein level for at least one or at least two genes, in particular at least two genes, selected from the group formed by TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1, preferably as listed in FIG. 1.

Embodiments described in this text in relation to the steps of providing a sample and measuring protein levels, are also embodiments in a method of measuring protein levels in a sample as described herein.

In addition, a method for measuring protein levels of the invention may further comprise a step of: —measuring in a serum sample of said allograft recipient a serum creatinine level; —determining the glomerular filtration rate, preferably by intravenously injecting (i) inulin, (ii) inulin-analogs such as sinistrin or (iii) radioactive substances for use in determining glomerular filtration rate, such as 51Cr-EDTA or 99mTc-DTPA, into the bloodstream of an allograft recipient and measuring its clearance; and/or assaying a urine sample of the allograft recipient for proteinuria by performing a dipstick test and/or by determining a human serum albumin (HSA) level through the use of liquid crystals and comparing such a HSA level to a reference value.

The invention further provides medical treatments for allograft recipients that are typed, or assigned, with a method of the invention. Since it is now possible to stratify allograft recipients according to a transplant rejection phenotype, therapy can be tailored to the needs of the patient. Further, the Examples indicate that a new group of patients, previously not identified with classical histological analysis of an allograft biopsy, is now identified and can be subjected to personalized therapy.

To that extent, the invention provides a standard-of-care therapeutic agent for use in the treatment of an allograft recipient suffering from, or undergoing, transplant rejection associated with an ABMR, wherein (i) said recipient, or a sample thereof, is typed as having, or undergoing, an ABMR according to a method of typing of the invention, or (ii) said recipient, or a sample thereof, is assigned to the ABMR group according to a method of assigning according to the invention.

The term “standard-of-care therapeutic agent”, as used herein, refers to a therapeutic compound, or a combination of such compounds, that is/are considered by medical practitioners as appropriate, accepted, and/or widely used for a certain type of patient, disease or clinical circumstance such as transplant rejection. Standard-of-care therapies for counteracting transplant rejection are available in the art. Specific standard-of-care therapeutic agents for use in the treatment of allograft recipients suffering from, or undergoing, transplant rejection associated with ABMR, include corticosteroids, rituximab, intravenous immunoglobulin (IVIG) products, bortezomib, eculizumab or combinations thereof. More generally, standard-of-care therapeutic agents in the treatment of transplant rejection include cyclosporine, tacrolimus, mycophenolic acid, sirolimus, everolimus, belatacept, basiliximab and antithymocyte globulin.

In the same manner, the invention also provides a method for treating an allograft recipient suffering from transplant rejection associated with an ABMR, comprising the steps of: —administering a therapeutically effective amount of a standard-of-care therapeutic agent to an allograft recipient suffering from, or undergoing, transplant rejection associated with an ABMR, wherein (i) said recipient, or a sample thereof, is typed as having, or undergoing, an ABMR according to a method for typing of the invention, or (ii) said recipient, or a sample thereof, is assigned to the ABMR group according to a method for assigning of the invention.

The term “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic agent. It is within the knowledge and capabilities of the skilled practitioner to determine therapeutically effective dosing regimens.

The term “administering”, as used herein, refers to the physical introduction of an agent or therapeutic compound to an allograft recipient patient, using any of the various methods and delivery systems known to those skilled in the art. The skilled person is aware of suitable methods for administration and dosage forms. Administration of small molecules can generally be performed by non-parenteral administration such as by oral and enteral administration. Preferred route of administration for protein-based agents such as antibodies is by parenteral administration, including intravenous, intramuscular, subcutaneous, intraperitoneal, spinal or other parenteral routes of administration, executed inter alia by injection or infusion in the form of a solution. Administering can be performed, for example, once, a plurality of times, and/or over one or more extended periods of time.

In the same manner, the invention also provides a use of a standard-of-care therapeutic agent in the manufacture of a medicament for treating an allograft recipient suffering from transplant rejection associated with an ABMR, wherein (i) said recipient, or a sample thereof, is typed as having, or undergoing, an ABMR according to a method for typing of the invention, or (ii) said recipient, or a sample thereof, is assigned to the ABMR group according to a method for assigning of the invention.

Preferably, in a medical method for treating transplant rejections associated with ABMR, the standard-of-care therapeutic agent is selected from the group formed by corticosteroids, rituximab, intravenous immunoglobulin (IVIG) products, bortezomib, eculizumab and combinations thereof; wherein said agent is for administration according to a therapeutically effective dosing regimen. More preferably, the standard-of-care therapeutic agent is bortezomib, eculizumab or rituximab.

For the purpose of clarity and a concise description, features are described herein as part of the same or separate aspects and preferred embodiments thereof, however, it will be appreciated that the scope of the invention may include embodiments having combinations of all or some of the features described.

The content of the documents referred to herein is incorporated by reference.

The invention will now be illustrated by the following Figure legends and Examples, which are provided by way of illustration and not of limitation and it will be understood that many variations in the methods described and the amounts indicated can be made without departing from the spirit of the invention and the scope of the appended claims.

### FIGURE LEGENDS

FIG. 1. List of biomarkers segregating ABMR from non-ABMR in renal allograft recipients.

Top 21 of selected upregulated proteins that segregate ABMR from non-ABMR phenotypes in step 1 and 2 (training cohort) in a case-control setup. The six proteins selected in green (grey) are the proteins for which two unique peptides (vide Table 1) were used to train and validate a statistical SVM model (Example 1). All 21 proteins are upregulated in AMBR cases (minimal fold change in log 2 is 0.8 in step 1 or step 2).

FIG. 2. ROC curve of validation dataset.

Receiver operating characteristic (ROC) curve obtained by employing the six proteins (Example 1)—detected in the form of the twelve peptides listed in Table 1—as biomarkers in a validation cohort (N=240) comprising patients having received a kidney allograft.

FIG. 3. Study outline

Study outline, showing the training (step 1 and 2) and validation cohorts (step 3).

FIGS. 4 and 5. ROC curves on training data set (FIG. 4) and on validation data set (FIG. 5).

Diagnostic accuracy of the protein biomarkers in the training and the validation set (Example 2). Receiver Operating Characteristic (ROC) curves are shown for the full model with 10 proteins (Table 4) for the training set (N=249; FIG. 4)) and for the validation set (N=391; FIG. 5). In the training dataset, the full model with 10 proteins has 98% AUC. In the validation dataset, this model has 88% AUC.

FIG. 6. Classification with two random proteins of Table 5.

Number of random proteins of Table 5 needed to achieve ABMR classification.

### EXAMPLES

### Example 1. Training and Validation of Biomarkers Segregating Antibody-Mediated Kidney Allograft Rejection from Other Kidney Allograft Rejection Phenotypes

Materials and Methods

Study Population

We performed a multicentre retrospective study. Patient who received a kidney allograft in four European clinical centres (University Hospital Leuven, Paris Necker, CHU Limoges and Hannover Medical School), were included in the study with written informed consent. Protocol or indication biopsies were performed and urine samples were collected. In this proteomics study, only urine samples were used for analysis. Biopsies were read by local and central pathologists, who classified all samples in four different phenotypes: Normal (NL), antibody mediated rejection (ABMR), T-cell mediated rejection (TCMR) and interstitial fibrosis and tubular atrophy (IFTA). Combinations of phenotypes were also possible.

General Study Design

The present study can be generally divided in three steps. In step 1, 130 urine samples of kidney allograft recipients were analysed, and in step 2 urine samples of 133 kidney allograft recipients were analysed. Steps 1 and 2 relate to the identification, and training, of the differentially expressed proteins for use as diagnostic ABMR biomarkers. Step 3 relates to independent validation of the biomarkers, in which step the diagnostic performance of the biomarkers was tested on 240 samples of kidney allograft recipients.

Urine Collection

Urine samples were collected in the four different clinical centres. Fresh urine samples, preferably the second voiding, were collected in the morning before the biopsy was taken. Urine creatinine, haemoglobin, leucocytes, glucose and protein content was measured locally using dipstick tests. Urine samples were centrifuged at 2,000 g at 4° C. for 20 minutes to remove cell debris and casts within 2 h after collection. The supernatant was stored below −20° C. until shipment to the analytical centres. Upon arrival, the samples were stored at −80° C.

Per step, samples were randomized in batches of 24 samples, taking into account that all batches contained samples from every clinical centre and all different phenotypes.

Sample Preparation

After a first concentration determination using the Pierce™ BCA Protein Assay Kit (Thermo Scientific), 2 mg of protein was processed on an Amicon Ultra-0.5 Centrifugal Filter Unit with a 10 kDa molecular weight cut-off membrane (Merck Millipore). The protein concentration of the concentrated samples was again determined using the same BCA Protein assay. Subsequently, 100 μg of protein was loaded on the Pierce™ Albumin Depletion Kit (Thermo Scientific) spin columns to deplete the samples from human albumin. After albumin depletion, the protein concentration was determined a last time. 20 μg of protein was denatured in 0.1% Rapigest (RapiGest™ SF, Waters). After denaturation, proteins were reduced by adding 2 μl of 200 mM TCEP (Tris(2-carboxyethyl)phosphine; Thermo Scientific) and incubating the sample for 1 h at 55° C. Afterwards, samples were alkylated by adding 2 μl of 375 mM IAA (Iodoacetamide; Thermo Scientific) for 30 minutes at room temperature protected from light. To precipitate the proteins, 1 ml of pre-chilled acetone was added and incubated overnight at −20° C. After a centrifugation step (10,000 g, 15 min., 4° C.), the protein pellet was resuspended in 20 μl 200 mM TEAB (Triethylammonium bicarbonate; Sigma-Aldrich). 1 μg of trypsin (Trypsin Gold, Mass Spectrometry Grade; Promega) was added to digest the proteins while incubating overnight at 37° C. The digestion was stopped and Rapigest was hydrolyzed by adding HCl to a final concentration of 200 mM (30 min. at room temperature). After a centrifugation step (10,000 g, 15 min., 4° C.), the pellet was removed and the samples were diluted in 2% acetonitrile, 0.1% formic acid to a final concentration of 0.2 μg/μl. All samples were spiked with 4 fmol/μl GFP ([Glu1]-Fibrinopeptide B human; Sigma-Aldrich).

Nano Reversed Phase Liquid Chromatography and Mass Spectrometry

In total, 1 μg of the peptide mixture, spiked with 20 fmol GFP, was loaded on the LC column. The tryptic peptide mixture was analysed on a Nano Acquity Ultra Performance LC system (Waters) using a nanoACQUITY UPLC Symmetry C18 Trap Column (180 μm×20 mm; Waters) coupled to a ACQUITY UPLC Peptide BEH C18 nanoACQUITY column (100 μm×100 mm; Waters). A linear gradient of mobile phase B (98% acetonitril, 0.1% formic acid, pH=2) from 5 to 45% in 68 min. was followed by a steep increase to 90% mobile phase B in 3 minutes. The flow was set at 400 nl/min. The nano-LC was coupled online to the LTQ Velos Orbitrap mass spectrometer (Thermo Scientific) via the nanospray ion source (Thermo Scientific).

The LTQ Velos orbitrap was set up in MS/MS shotgun mode, where a full MS1 precursor scan (300-2000 m/z, resolution 60,000) was followed by a maximum of 10 collision induced dissociation (CID) MS2 spectra of the 10 most intense precursor peaks. CID spectra were obtained in the linear ion trap of the mass spectrometer. The normalized collision energy used in CID was set at 35%. We applied a dynamic exclusion of 30 s for data dependent acquisition.

Quality Control Analysis

The MS/MS results (raw data) together with the Proteome Discoverer results were inspected in a quality control (QC) analysis. QC analysis is done systematically as it guarantees the quality of the sample and the MS instruments at each moment for each sample. If for several reasons, samples do not meet the requested QC parameters, these samples are excluded for further data analysis steps.

Data Enrichment Process

To get quantitative data on all peptides for each sample, we developed in house software to look up peak intensities in the raw MS1 data. In short, this software tool looks up m/z values in raw MS1 data with a delta ppm of 5 in a retention time window of 10 minutes. That way, a data matrix is obtained containing quantitative information from almost all identified peptides. The algorithm also cleans the resulting data by using a decoy search and also peak shape is checked.

Model Building

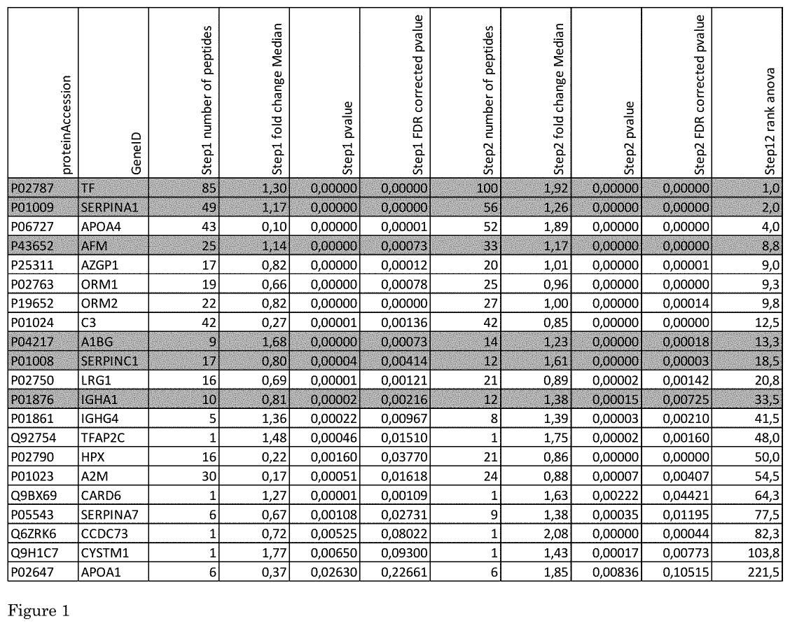

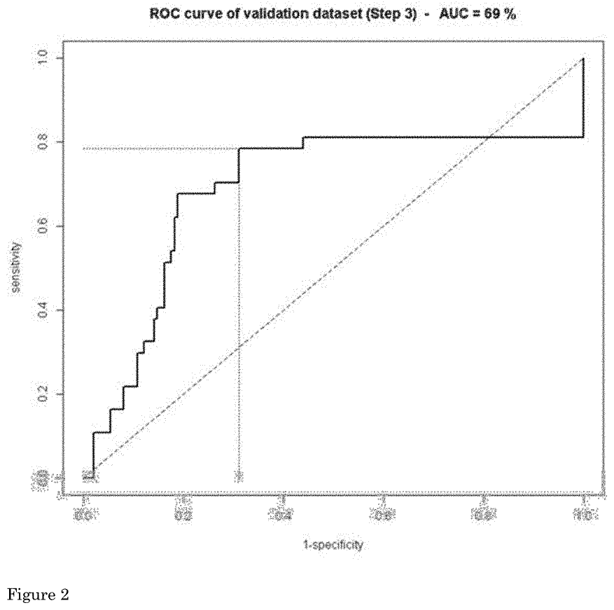

Data of step 1 and step 2 were used as training data. Step 1 data was used for selecting the significantly differentially expressed proteins. Step 2 data was used as a first verification dataset. The hypothesis we tested was ABMR vs. no-ABMR. ANOVA was used to select proteins that were significantly upregulated or downregulated in ABMR cases. ANOVA was applied on generated data of each protein of step 1 and step 2 samples independently. In the final list, the top 21 proteins were selected that are upregulated in ABMR1 compared to ABMR0 (i.e. ABMR vs. no-ABMR) with at least a fold change of 0.8 (log 2 fold change) in one of the two steps (on the protein level) (FIG. 1).

Once the proteins were selected, we selected 2 unique peptides per protein (no miscleavages) (Table 1). The selection was based on results in peak scoring and their suitability for targeted analysis. If a protein in the final list does not have two peptides fulfilling those criteria, the protein is left out of the model. This way, the model can be validated in an extra validation step following this study, using targeted proteomics. Only unique peptides were used in this ANOVA analysis. Results of step 1 and step 2 analyses are listed by log 2 fold change and p-value in Table 1 below.

Model Validation

Finally, the support vector machine (SVM) that was modelled, is applied on the normalized, enriched data obtained from the step 3 samples, which serve as an independent validation dataset. An ROC curve is generated and the results are inspected and plot per individual patient.

Results

Peptide Identification

All data was searched against the human Uniprot database using Proteome Discoverer software (version 2.1; Thermo Scientific). Both search engines Mascot and Sequest were used. The following search parameters were used: precursor mass tolerance 10 ppm, fragment mass tolerance 0.5 Da. Trypsin was chosen as the cleavage enzyme and 2 missed cleavages were allowed. Carbamidomethylation was set as a fixed modification on cysteine and methionine oxidation and serine, tyrosine and threonine phosphorylation were set as variable modifications. The resulting peptide identification results were filtered using the following settings: only the high confident with a False Discovery Rate (FDR)<5% based on the target-decoy approach and the first ranked peptides were included, to yield the proteins indicated in FIG. 1.

In an alternative experimental set-up, it was established that the expression products of the genes listed in FIG. 1 do not provide for differentiation between ABMR and non-ABMR phenotypes when said expression products are mRNA (data not shown).

Statistical Analysis—Model Building

ANOVA was applied on the data obtained in step 1 and step 2, and results were listed by p-value. The top 21 selected proteins are shown in FIG. 1. We selected two unique peptides with high confident identification per protein that can also be used in targeted analysis. If such unique peptides were not available for a listed protein, the protein was not used in the model. This way, our final model contained 12 peptides from 6 proteins. The selected peptides are shown in Table 1 below. Biopsy results are considered as “outcome variable” in our analysis. We trained a support vector machine on the data of step 1 and step 2 using the 12 selected peptides as parameters. After fixing the cutoff point, we obtained a sensitivity of 84.7% and a specificity of 78.3% (vide Table 2).

TABLE 1
Selected list of unique peptides used for training the SVM model.

| | Protein | | | | | | |
|---|---|---|---|---|---|---|---|
| | Accession | | Step1_ | Step2_ | Step1_ | Step2_ | SEQ |
| GeneID | No. | Sequence | foldchange | foldchange | pvalue | pvalue | ID |
| SERPINC1 | P01008 | EQLQDMGLVDLFSPEK | 1.23 | 1.58 | 0.00257 | 0.00007 | 1 |
| SERPINC1 | P01008 | VAEGTQVLELPFK | 1.40 | 1.64 | 0.17546 | 0.00013 | 2 |
| SERPINA1 | P01009 | LSITGTYDLK | 1.30 | 1.63 | 0.00027 | 0.00039 | 3 |
| SERPINA1 | P01009 | SVLGQLGITK | 1.26 | 1.36 | 0.00017 | 0.00012 | 4 |
| IGHA1 | P01876 | DASGVTFTWTPSSGK | 1.14 | 1.71 | 0.00081 | 0.00006 | 5 |
| IGHA1 | P0187G | TFTcTAAYPESK | 0.86 | 2.01 | 0.00145 | 0.00002 | 6 |
| TF | P02787 | cSTSSLLEAcTFR | 1.94 | 3.00 | 0.00001 | 0.00000 | 7 |
| TF | P02787 | DSGFQMNQLR | 1.54 | 2.11 | 0.00018 | 0.00002 | 8 |
| A1BG | P04217 | ATWSGAVLAGR | 1.97 | 1.30 | 0.00002 | 0.02962 | 9 |
| A1BG | P04217 | cEGPIPDVTFELLR | 1.91 | 1.96 | 0.00003 | 0.01734 | 10 |
| AFM | P43652 | AESPEVcFNEESPK | 1.54 | 1.66 | 0.00153 | 0.00001 | 11 |
| AFM | P43G52 | FTDSENVcQER | 1.34 | 1.71 | 0.00076 | 0.00001 | 12 |

TABLE 2
Contingency table for the training dataset.

| | | biopsy diagnosis | | |
|---|---|---|---|---|
| | | no ABMR | ABMR | |
| model | no ABMR | 160 | 13 | 173 |
| classification | ABMR | 29 | 47 | 76 |
| on | | 189 | 60 | 249 |
| | | | sensitivity = | 84.7% |
| | | | specificity = | 78.3% |

Table 2 shows an overview of the samples classification using the model compared to the biopsy results. We see that for diagnosing ABMR, the model is more conservative than the pathologist's decision, because in almost 30% of the cases for which the biopsy results in the diagnosis of ABMR, the model disagrees, while for ABMR0 (i.e. no ABMR), the model only disagrees in 15% of the cases. However, these 15% are very important cases, because here the model could possibly pick up signs of ABMR before the histology reveals any signs of rejection. In the case of ABMR diagnosis, the model is envisaged to help pathologists make a more accurate diagnosis.

Statistical Analysis—Model Validation

The SVM model is fixed using data from step 1 and 2 as a training dataset. Thus the step 3 samples are used as a completely independent validation dataset. Validation took place in a large cohort (240) of independent patients that previously received a kidney allograft.

77% of the samples is correctly classified. After fixing the cutoff point, a sensitivity of 79.1% and a specificity of 70.3% was obtained (vide Table 3).

TABLE 3
Contingency table for the validation dataset
Validation data

| | | cutoff = 0.75 | | |
|---|---|---|---|---|
| | | biopsy diagnosis | | |
| | | no | ABMR | |
| | | ABMR | | |
| model | no | 117 | 11 | 128 |
| classification | ABMR | | | |
| | ABMR | 31 | 26 | 57 |
| | | 148 | 37 | 185 |

| | |
|---|---|
| sensitivity = | 79.1% |
| specificity = | 70.3% |

### Example 2

This Example builds on Example 1.

Additional renal allograft recipients were included in the study. Sample preparation and protein expression level measurement are as indicated in Example 1.

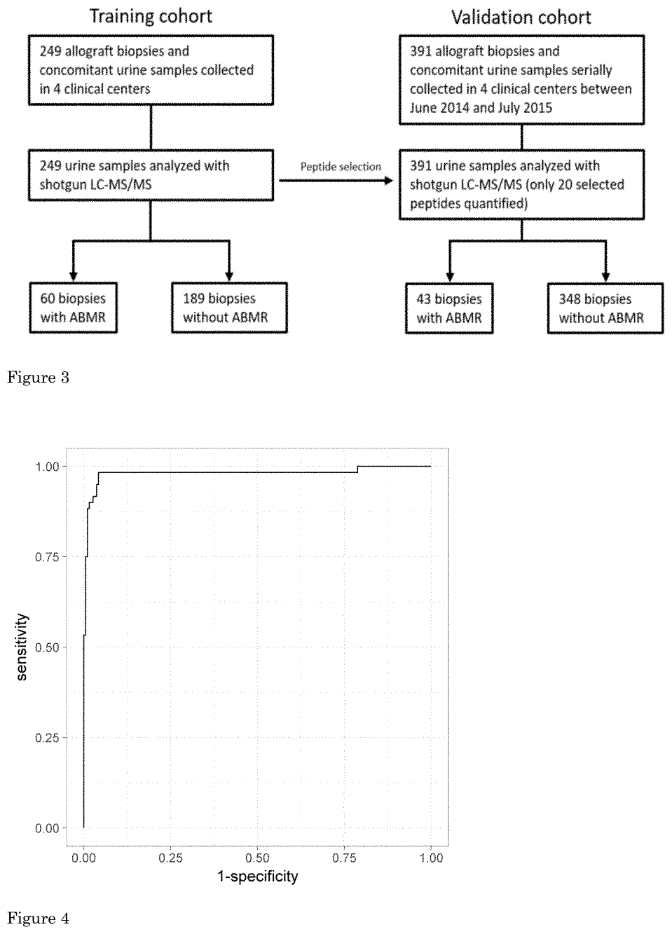

The training data set again represented 249 kidney allograft recipients, and the validation data set now represented 391 kidney allograft recipients. All biopsies included in this study were reviewed and graded in a blinded fashion by a central pathologist independent from the original center. Study outline is provided in FIG. 3.

Results

In the training set, 60/249 cases showed ABMR (24.1%), and 43/391 (11.0%) in the validation set.

Results of Example 1 were also achieved when expanding patient population.

Subsequently, in the same manner as in Example 1, a diagnostic model was build now on the basis of ten proteins (A1BG, AFM, APOA1, APOA4, IGHA1, IGHG4, LRG1, SERPINA1, SERPINC1 and TF) by selecting 2 unique peptides per protein. This set of 20 peptides is provided in Table 4 below.

TABLE 4
Selected list of peptides that were used for
training in the the SVM model training set.

| GeneID | peptide | SEQ ID |
|---|---|---|
| A1BG | ATWSGAVLAGR | 9 |
| A1BG | cEGPIPDVTFELLR | 10 |
| AFM | AESPEVcFNEESPK | 11 |
| AFM | FTDSENVcQER | 12 |
| APOA1 | DLATVYVDVLK | 13 |
| APOA1 | DYVSQFEGSALGK | 14 |
| APOA4 | ISASAEELR | 15 |
| APOA4 | SLAELGGHLDQQVEEFR | 16 |
| IGHA1 | DASGVTFTWTPSSGK | 5 |
| IGHA1 | TFTcTAAYPESK | 6 |
| IGHG4 | TTPPVLDSDGSFFLYSR | 17 |
| IGHG4 | YGPPcPScPAPEFLGGPSVFLFPPKPK | 18 |
| LRG1 | ALGHLDLSGNR | 19 |
| LRG1 | DLLLPQPDLR | 20 |
| SERPINA1 | LSITGTYDLK | 3 |
| SERPINA1 | SVLGQLGITK | 4 |
| SERPINC1 | ADGEScSASMMYQEGK | 21 |
| SERPINC1 | IEDGFSLK | 22 |
| TF | cSTSSLLEAcTFR | 7 |
| TF | DSGFQMNQLR | 8 |

The diagnostic performance of each of said ten genes is shown in Table 5.

TABLE 5
List of 10 proteins that segregate ABMR from
non ABMR phenotypes in the training data set.

| | | | | | | maximum | |
|---|---|---|---|---|---|---|---|
| | | | total | median | FDR | number of | |
| | | total | number | log2 fold | corrected | peptide | |
| | Uniprot | number of | of unique | change | p-value | spectrum | maximum |
| | protein | peptides | peptides | training | training | matches | sequence |
| GeneID | Accession | identified | identified | dataset | dataset | PSMs | coverage |
| A1BG | P04217 | 12 | 4 | 1.13 | 0.01093 | 227 | 54.14 |
| AFM | P43652 | 14 | 14 | 1.00 | 0.00005 | 55 | 37.06 |
| APOA1 | P02647 | 16 | 3 | 0.61 | 0.04509 | 54 | 60.30 |
| APOA4 | P06727 | 21 | 21 | 0.60 | 0.00005 | 148 | 70.20 |
| IGHA1 | P01876 | 12 | 4 | 0.87 | 0.00030 | 79 | 68.84 |
| IGHG4 | P01861 | 2 | 2 | 0.78 | 0.00757 | 50 | 39.45 |
| LRG1 | P02750 | 9 | 9 | 0.68 | 0.00000 | 86 | 44.96 |
| SERPINA1 | P01009 | 24 | 18 | 1.29 | 0.00000 | 1057 | 71.29 |
| SERPINC1 | P01008 | 9 | 7 | 0.86 | 0.00022 | 35 | 44.61 |
| TF | P02787 | 53 | 31 | 1.37 | 0.00000 | 1420 | 82.38 |

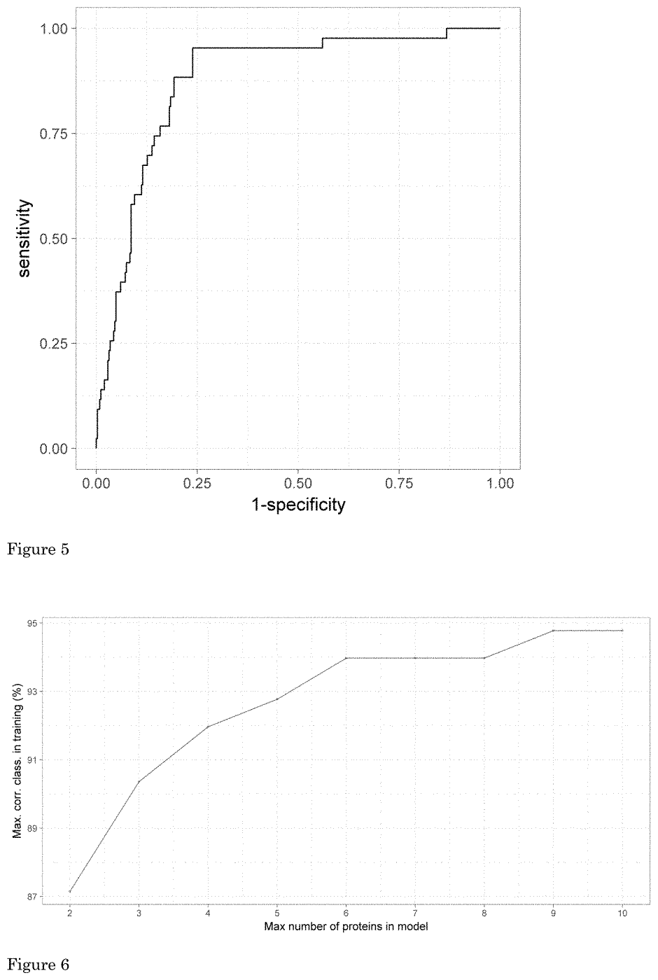

This set of ten proteins, each protein represented by two peptides, is considered a good representation of ten random proteins selected from FIG. 1. The ROC curve of the ten protein model is shown in FIG. 4 (training data set) and FIG. 5 (validation data set). After fixing the cutoff point to 0.30, this model reached a sensitivity of 95% and a specificity of 96%. In addition, it was further shown that the diagnostic performance of this ten protein model is also achieved with six random proteins of said set of 10 proteins (Tables 6 and 7).

TABLE 6
Different sets of proteins included in the model.

| model | model | Proteins included in the model | | | | | | | | | |
|---|---|---|---|---|---|---|---|---|---|---|---|
| name | description | A1BG | AFM | APOA1 | APOA4 | IGHA1 | IGHG4 | LRG1 | SERPINA1 | SERPINC1 | TF |
| model10 | all 10 | x | x | x | x | x | x | x | x | x | x |
| | proteins | | | | | | | | | | |
| model6A | First set of | x | | x | x | x | | | x | | x |
| | 6 proteins | | | | | | | | | | |
| model6B | Second set of | x | | | x | x | | x | x | | x |
| | 6 proteins | | | | | | | | | | |
| model6C | Third set of | x | | x | x | | | x | x | | x |
| | 6 proteins | | | | | | | | | | |

TABLE 7
Results for all 4 models fitted for the training dataset and the validation dataset.

| Model name | TP | TN | FP | FN | Sensitivity | Specificity | PPV | NPV |
|---|---|---|---|---|---|---|---|---|
| Training set | | | | | | | | |
| model10 | 57 | 182 | 7 | 3 | 0.95 | 0.96 | 0.89 | 0.98 |
| model6A | 57 | 179 | 10 | 3 | 0.95 | 0.95 | 0.85 | 0.98 |
| model6B | 57 | 178 | 11 | 3 | 0.95 | 0.94 | 0.84 | 0.98 |
| model6C | 57 | 178 | 11 | 3 | 0.95 | 0.94 | 0.84 | 0.98 |
| Validation set | | | | | | | | |
| model10 | 41 | 263 | 85 | 2 | 0.95 | 0.76 | 0.33 | 0.99 |
| model6A | 36 | 243 | 105 | 7 | 0.84 | 0.70 | 0.26 | 0.97 |
| model6B | 36 | 241 | 107 | 7 | 0.84 | 0.69 | 0.25 | 0.97 |
| model6C | 40 | 243 | 105 | 3 | 0.93 | 0.70 | 0.28 | 0.99 |

TP: true positives; TN: true negatives; FP: false positives; FN: false negatives; PPV: positive predictive value; NPV: negative predictive value.

Finally, the diagnostic performance of at least two random proteins of said set of 10 proteins is shown in FIG. 6. Unexpectedly, it was shown that at least two random proteins of said set of 10 already provide for a correlation with ABMR of >87%. It is shown that a plateau in diagnostic performance is achieved already with six proteins.

1. A method for typing an allograft recipient for the presence or absence of an antibody mediated rejection (ABMR), comprising the steps of
measuring in a sample comprising proteins from an allograft recipient a protein level for at least two genes selected from the group consisting of TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1;
comparing said measured protein level to a reference protein level for said at least two genes; and
typing said allograft recipient for the presence or absence of an ABMR on the basis of the comparison of the measured protein level and the reference protein level.

2. The method according to claim 1, wherein the method is for typing a sample of said allograft recipient for the presence or absence of an ABMR.

3. The method according to claim 1, wherein the allograft recipient is a renal allograft recipient.

4. The method according to claim 1, wherein the sample is a body fluid sample.

5. The method according to claim 1, wherein a protein level is measured for at least 6 genes selected from the group consisting of TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1.

6. The method according to claim 1, wherein a protein level is measured for at least two genes selected from the group consisting of A1BG, AFM, APOA1, APOA4, IGHA1, IGHG4, LRG1, SERPINA1, SERPINC1 and TF.

7. The method according to claim 6, wherein a protein level is measured for at least 6 genes selected from the group consisting of A1BG, AFM, APOA1, APOA4, IGHA1, IGHG4, LRG1, SERPINA1, SERPINC1 and TF.

8. The method according to claim 1, wherein said allograft recipient, or sample thereof, is typed as having an ABMR when said protein levels are increased as compared to a reference protein level for said at least two genes in a reference sample of an allograft recipient not having an ABMR.

9. The method according to claim 1, further comprising the steps of:
digesting proteins in said sample with trypsin so as to provide a mixture of peptides;
subjecting said mixture of peptides to a step of liquid chromatography so as to provide an eluate comprising peptides; and
performing a step of mass spectrometry on said eluate to measure a peptide level for at least two peptides, said peptide level for said at least two peptides representing the protein level for said at least two genes.

10. The method according to claim 9, wherein said at least two peptides are selected from SEQ ID NOs: 1-22.

11. The method according to claim 1, wherein measurement of said protein level is performed by an enzyme-linked immunosorbent assay (ELISA).

12. A method for assigning an allograft recipient to an ABMR group or a non-ABMR group comprising the steps of:
measuring in a sample comprising proteins from an allograft recipient suffering, or at risk of suffering, from transplant rejection a protein level for at least two genes selected from the group consisting of TF, SERPINA1, APOA4, AFM, AZGP1, ORM1, ORM2, C3, A1BG, SERPINC1, LRG1, IGHA1, IGHG4, TFAP2C, HPX, A2M, CARD6, SERPINA7, CCDC73, CYSTM1 and APOA1;
comparing said measured protein level to a reference protein level for said at least two genes; and
assigning said allograft recipient to said ABMR group or to said non-ABMR group on the basis of the comparison of the measured protein level and the reference protein level.

13. The method of claim 1 wherein the sample is a urine sample and at least two proteins from the at least two genes serve as a marker for the presence or absence of an ABMR in an renal allograft recipient.

14. A standard-of-care therapeutic agent for use in the treatment of an allograft recipient suffering from transplant rejection associated with an ABMR, wherein said recipient, or a sample thereof, is typed as having an ABMR according to the method of claim 1.

15. The standard-of-care therapeutic agent for use according to claim 14, wherein said agent is selected from the group consisting of corticosteroids, rituximab, intravenous immunoglobulin (IVIG) products, bortezomib, eculizumab and combinations thereof; and wherein said agent is for administration according to a therapeutically effective dosing regimen.

16. A method for treating an allograft recipient suffering from transplant rejection associated with an ABMR, comprising the steps of:
administering a therapeutically effective amount of a standard-of-care therapeutic agent to an allograft recipient suffering from transplant rejection associated with an ABMR, wherein said recipient, or a sample thereof, is typed as having an ABMR according to the method of claim 1.

17. A standard-of-care therapeutic agent for use in the treatment of an allograft recipient suffering from transplant rejection associated with an ABMR, wherein said recipient, or a sample thereof, is assigned to the ABMR group according to a method of claim 12.

18. The standard-of-care therapeutic agent for use according to claim 17, wherein said agent is selected from the group consisting of corticosteroids, rituximab, intravenous immunoglobulin (IVIG) products, bortezomib, eculizumab and combinations thereof; and wherein said agent is for administration according to a therapeutically effective dosing regimen.

19. A method for treating an allograft recipient suffering from transplant rejection associated with an ABMR, comprising the steps of:
administering a therapeutically effective amount of a standard-of-care therapeutic agent to an allograft recipient suffering from transplant rejection associated with an ABMR, wherein said recipient, or a sample thereof, is assigned to the ABMR group according to a method of claim 12.

20. The method of claim 19 wherein the standard-of-care therapeutic agent is selected from the group consisting of corticosteroids, rituximab, intravenous immunoglobulin (IVIG) products, bortezomib, eculizumab and combinations thereof; and wherein said agent is for administration according to a therapeutically effective dosing regimen.
